(19) United States
(12) Patent Application Publication
(10) Pub. No.: US 2020/0197039 A1
(43) Pub. Date: Jun. 25, 2020

(54) DISSECTING DEVICE

(71) Applicant: Robert S. Hatch, Ormond Beach, FL (US)

(72) Inventor: Robert S. Hatch, Ormond Beach, FL (US)

(21) Appl. No.: 16/720,188

(22) Filed: Dec. 19, 2019

Related U.S. Application Data
(60) Provisional application No. 62/781,910, filed on Dec. 19, 2018.

(51) Int. Cl. A61B 17/32

(52) U.S. Cl. CPC A61B 17/320016; A61B 2017/0414; A61B 2017/320044

(57) ABSTRACT

In embodiments herein, a non-invasive surgical trigger finger repair device is provided. The device includes a proximal end including a component for coupling to a filament, a blunted distal end, an elongated straight section extending from the distal end toward the proximal end, and a curvilinear section between the elongated straight section and the proximal end, wherein manipulation of the device and targeting of the distal end occurs by movement of the curvilinear section and/or the proximal end.

### BACKGROUND

Surgery frequently involves cutting or stitching pathologic tissues lying below the skin. In order to access these tissues, an incision is usually made, and dissection continues to the depth of the targeted pathological tissue. The length of the incision is generally proportional to the depth and size of the target tissue. Dissection to the pathology disrupts healthy surrounding tissue, exposing the patient to substantial risk of surgical complications such as bleeding, nerve injury, scar tissue development, joint contracture, painful neuroma, and prolonged healing time.

Recent developments in real time imaging technologies, such as ultrasound and digital flouroscopy, enable deep tissue visualization without disruption of the more superficial and surrounding healthy tissues. Pathologic processes are viewed from a distance and without harming nonpathologic structures. With improvements in modern imaging have come methods and apparatuses for accessing and manipulating the target deep tissue while only minimally disturbing the more superficial tissues. These types of procedures can substantially decrease morbidity associated with procedures that compromise a greater volume of tissue. Recent trends for modern surgery have been toward less invasive procedures that meet or exceed current gold standard results for established open procedures.

Traditional straight needles having no lengthwise curvature have been the workhorse procedural tool for accessing deep musculoskeletal tissues by ultrasound guidance. Needling procedures remain useful primarily for purposes of injection to deliver necessary medications to surrounding tissues for enhanced healing, for example. The injectate most commonly contains anti-inflammatory medicine, biologically active materials or sclerosing agents. Injection can also be used mechanically for dissection by forcefully injecting fluid along a tissue plane, i.e. hydrodissection. Needling techniques can induce small injuries into pathologic tissue to stimulate a healing response or disrupt tissues therapeutically.

More recently small gauge cutting instruments have been inserted to cut tissues such as the transverse carpal ligament for treatment of carpal tunnel syndrome and the A1 pulley to treat trigger finger, in some examples. Advancements in minimally invasive, ultrasound-guided injection and cutting techniques may be used to decrease the length of a skin incision, for example. In some non-limiting examples, the length of a standard open carpal tunnel surgery incision may include between 2-3 centimeters or more as compared to some ultrasound-guided methods having incisions 5 mm or less. Minimally invasive, also called “ultra minimally invasive” procedures often feature smaller dissection and advanced imaging-based technologies (e.g. arthroscopic, ultrasound, fluoroscopic etc.) with unique instrumentation. In some non-limiting embodiments, these procedures may include skin incisions that are less than or equal to 5 mm. Accomplishing a desired surgical goal while minimizing disruption of adjacent tissues substantially reduces recovery times, post-operative pain and narcotic use.

Acquired trigger finger is an infirmity of the hand where the relationship between a ligament (usually the A1 pulley) and the flexor tendons produce an inefficiency of movement whereby contact pressures between the structures are too high. Flexor tendons connect muscle to bone and move the joints of the digit that the tendon spans. For smooth motion, there must be no obstruction impacting the course of the tendon. A variety of tendon-related pathologies increase friction between the tendon and the overlying A1 pulley. The most common pathology involves a pathologic swelling of the tendon and its synovium. The resultant enlarged size of the tendon prevents it from sliding smoothly along its course. The enlarged tendon causes increased friction resulting in pain to the subject and ultimately reduces the effective speed of action of the tendon. These factors may result in a clicking/popping sensation as the inflamed area passes by an overlying ligamentous sheath (the A1 pulley) during muscle-tendon action. When the condition is severe there may be inhibited motion between the tendon and the ligament. As a result, the digit becomes locked in a position of flexion.

Treatments for trigger finger are commonly directed to the tenosynovium or to the A1 pulley. For severe or resistant conditions, surgical solutions are indicated.

There are many surgeries for trigger finger, all having the purpose of decreasing the pathologic friction. Most surgeries for trigger finger cut the A1 pulley to decrease contact pressures on the underlying flexor tendon. In some instances, an open release is used wherein a small incision (in some examples up to a 2 cm incision) is made by a scalpel followed by dissection to the A1 pulley. The A1 pulley is then cut under direct vision. These open procedures are often done in an operating room or an ambulatory surgery center and may utilize other expensive resources such as an anesthesiologist. This procedure may allow for transection of the A1 pulley but the overlying fibrofatty soft tissue and skin are also cut in the process, which may cause post-operative complications such as wound dehiscence, infection, scar tissue related contractures and pain, among others.

### BRIEF DESCRIPTION

A more particular description briefly stated above will be rendered by reference to specific embodiments thereof that are illustrated in the appended drawings. Understanding that these drawings depict only typical embodiments and are not therefore to be considered to be limiting of its scope, the embodiments will be described and explained with additional specificity and detail through the use of the accompanying drawings in which:

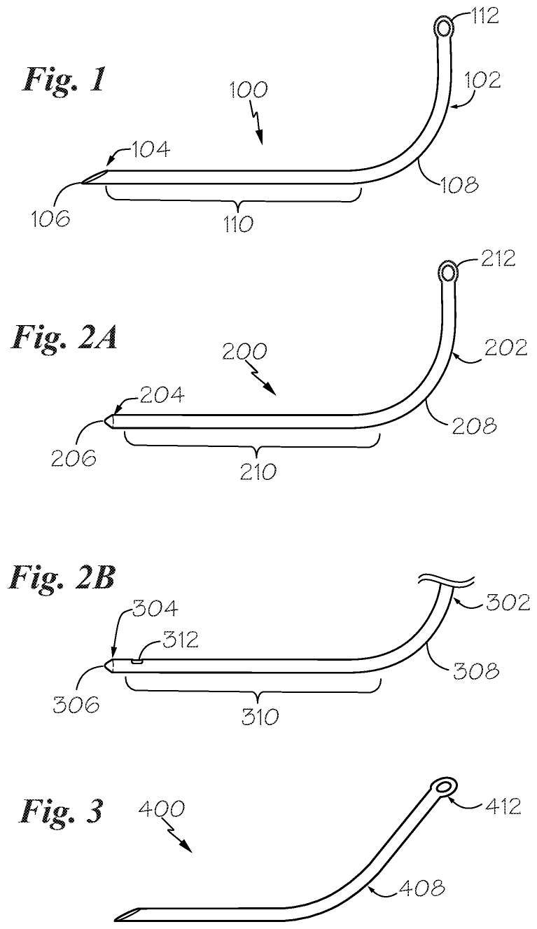

FIG. 1 is a side view of an embodiment of a device described herein.

FIG. 2A is a side view of another embodiment of a device as described herein.

FIG. 2B is a side view of yet another embodiment of a device as described herein.

FIG. 3 is a side view of another embodiment of a device.

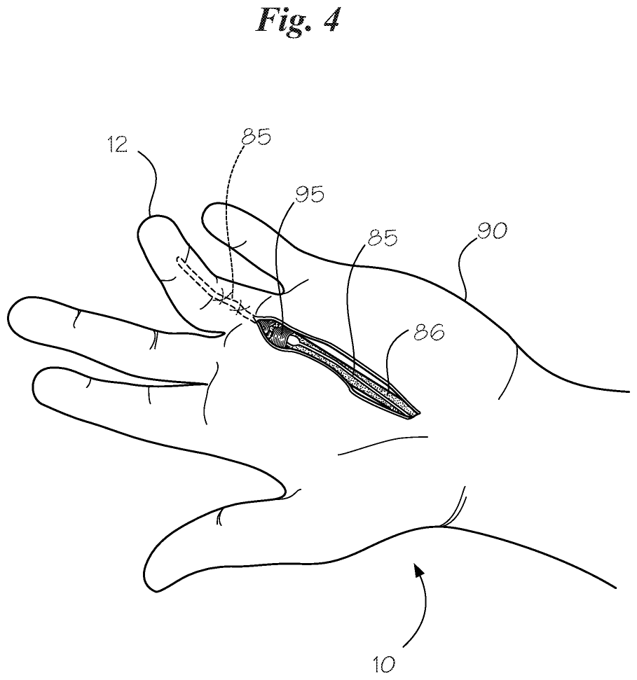

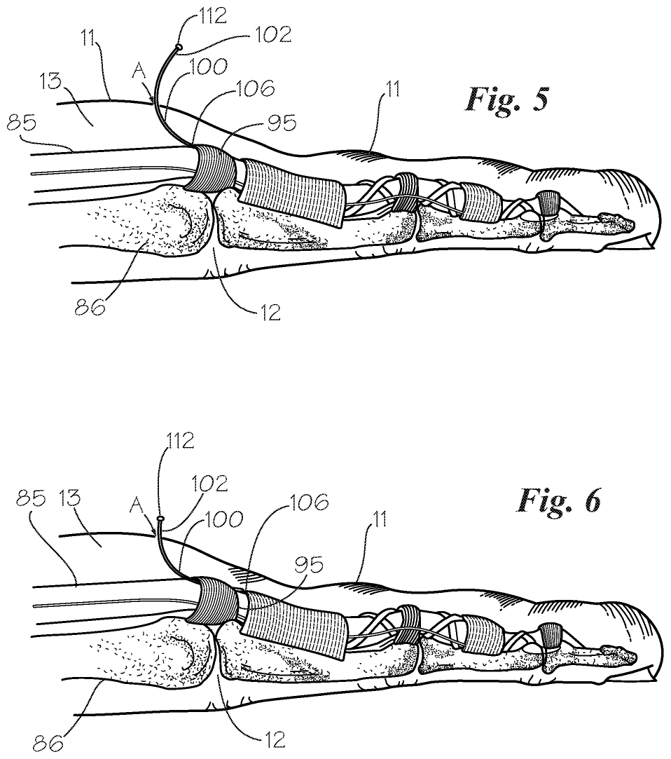

FIG. 4 is a perspective view of a hand demonstrating flexion as a result of trigger finger.

FIGS. 5-13 include step by step illustrations of the method of treating trigger finger using one embodiment of a device according to one method described herein.

### DETAILED DESCRIPTION

### Definitions

The term “filament” as used herein includes, but is not limited to sutures, or cutting wire or dilating implement/dissecting device etc.)

The term “curvilinear” as used herein, includes a segment of a section of the needle or apparatus forming at least a partially curved or arced portion of the needle or apparatus. The curvilinear portion of the needle or apparatus may be used to create a torque of the needle within a tissue, in one non-limiting example.

The term “torque” as used herein, includes a motion or force that causes rotation, or the application of a twisting force to an object. In one non-limiting example described herein, a torque occurs to the needle or apparatus by manipulating a proximal end of the needle or apparatus via the curvilinear section, to twist or rotate the needle or apparatus to displace a distal end of the needle or apparatus. Torque can be described as placing force on one end of the device, causing pivoting of the device, forcing movement of the distal end of the device.

The term “trailing filament” or “filament” as used herein, may include, sutures and the like, dilating devices, cutting wire, debriding devices, diagnostic/sensory equipment, bacteriocidal gauze, hemostatic materials, and the like.

It is to be noted that the terms “first,” “second,” and the like as used herein do not denote any order, quantity, or importance, but rather are used to distinguish one element from another. The terms “a” and “an” do not denote a limitation of quantity, but rather denote the presence of at least one of the referenced item. Furthermore, to the extent that the terms “including,” “includes,” “having,” “has,” “with,” or variants thereof are used in either the detailed description and/or the claims, such terms are intended to be inclusive in a manner similar to the term “comprising.” The modifier “about” used in connection with a quantity is inclusive of the stated value and has the meaning dictated by the context (e.g., includes the degree of error associated with measurement of the particular quantity). It is to be noted that all ranges disclosed within this specification are inclusive and are independently combinable.

The terminology used herein is for the purpose of describing particular embodiments only and is not intended to be limiting. As used herein, the singular forms “a,” “an,” and “the” are intended to include the plural forms as well, unless the context clearly indicates otherwise these terms do not denote a limitation of quantity, but rather denote the presence of at least one of the referenced item. Furthermore, to the extent that the terms “including,” “includes,” “having,” “has,” “with,” or variants thereof are used in either the detailed description and/or the claims, such terms are intended to be inclusive in a manner similar to the term “comprising.” Moreover, unless specifically stated, any use of the terms first, second, etc., does not denote any order, quantity or importance, but rather the terms first, second, etc., are used to distinguish one element from another.

### Overview

Trigger finger may be treated by minimally invasive surgical techniques. Minimally invasive or percutaneous procedures have been developed to address the trigger finger problem for a variety of reasons, including to attempt reduction of the overall cost of the procedure, to decrease the dissection-related tissue insult/injury, to increase the speed of the procedure, and to decrease the time for recovery. However, most efforts to design a device that transects the A1 pulley are of an elongated tool having a handle at one end and a cutting edge at the other. No suture needle design for the purpose of cutting the A1 pulley has been presented.

In some embodiments herein, a versatile apparatus for cutting deep tissue without making an incision is provided. The device and method embodiments described herein result in decreased or elimination of destruction of healthy tissue during a trigger finger repair surgery. By use of the device embodiments described herein, nonpathological tissues are nominally disturbed thereby decreasing healing time and complications. In addition, overall speed and efficiency of the procedure is improved by embodiments described herein that can be performed in a clinical setting with a patient under local anesthesia and without a tourniquet.

Embodiments described herein include a device which can be used to percutaneously navigate a desired anatomic path or can be used to place a filament or other dissecting instrument precisely against anatomy to cut, stitch, debride, dilate or guide, for example. In some embodiments, the device may include a cutting edge and in one example, no trailing filament or suture would be required. “Incisionless procedures” refer to a subclass of percutaneous procedures whereby only a puncture, usually requiring no stitching, is made at an exposed anatomic surface for the purpose of accessing and/or manipulating deeper tissues for a medical purpose and treatment. Furthermore, it should be understood that the exposed tissue can be a location that is first revealed during an open procedure, or by trauma, whereby a percutaneous method as described herein, using an embodiment of a device as described herein, may be used to efficiently continue or complete the surgical procedure.

Advanced imaging may improve tracking of an instrument maneuvering through anatomy. In embodiments described herein, the device may include a sharp or blunted end, or ends to navigate the desired tissues. The sharp version is designed to pierce neatly through skin and dense tissue. Between the extremes of razor sharpness and nonpenetrating dullness lies an intermediate range that can otherwise be manufactured to suit the surgical effort. As such, duller embodiments effectively navigate through tissue, and through the potential spaces between tissues, but cannot easily pierce healthy skin. Moreover, there may be embodiments with completely dull ends, which navigate through tissue and spaces in between, but cannot pierce skin. Embodiments of the device described herein may also be used to join predrilled bone fragments. For soft tissue and bone, the device can be trailed by a filament, including, but not limited to a flexible wire or other attachable filament device having a suitable geometry and structure depending upon the requirements of the intervention.

Ideally, advanced imaging ensures unintentional errant placement of a cutting instrument which can harm and even irreversibly injure tissue. Present day ultrasound technologies can easily detect a needle-like object of sufficient length. Other geometries can be added to a portion of the device to further reflect sound waves to improve detection by the machine such as a flat surface exposed to the sound waves or other configurations that enhance sound wave reflection back to the machines detection system. In some embodiments, these geometries may be added to a straight portion of the device.

Prior art designs may include a traditional needle used to dive into tissue and then move through the tissue to return to a surface point where the tip or sharp end portion of the needle can be seen and then manually retrieved. In contrast, embodiments described herein provide a uniquely structured needle that may lead, in some embodiments, by a straight segment whereby the device can be inserted through skin, whereby it traverses through complex tissue planes and is returned to a surface at another point, in one non-limiting embodiment.

In some embodiments described herein, the body of the device may be constructed from a relatively stiff and sturdy material. Such materials may include, but are not limited to, metal, thermoplastic materials and ceramics, for example. Any other materials known to those skilled in the art may also be used. Embodiments wherein the device comprises a blunted end may not require materials that require grinding manufacturing techniques. In some embodiments provided herein, the device may include a proximal end, a distal end, a curvilinear segment, and a substantially straight and linear distal segment. Some embodiments may include an attachment component for other surgical tools. Some embodiments may include several sites for attachment for sutures, dilating tools, debriding tools and the like. The proximal end may include a region suitable for handling by an operator, for example, and may include a degree of curvature required for return of the distal end of the device to the skin surface. In one example, the proximal end is of a length and size for manipulation by a user. In a non-limiting embodiment, at the distal end of the device, a sharp distal tip may be provided.

The distal tip of the device is the terminal portion of the substantially straight segment which ends at the first deflection of the device into a second axis. If the straight proximal segment of the device aligns with the “X” axis (of a Cartesian plane) then at least a portion of the proximal (curvilinear) portion of the device aligns with the “Y”-axis. The proximal curvilinear portion may be at least 5 mm or larger in one non-limiting embodiment. In another non-limiting embodiment, the curvilinear portion may be at least 3 mm or larger in another non-limiting embodiment. The proximal section is joined to the substantially straight section by the curvilinear section, in a gradual continuous arced profile, in one embodiment.

A “Z” axis measurement could be applicable for “out of plane” proximal-distal segment alignment and for an altered or dilated opening, in some instances termed a “needle eye” which may protrude substantially along the “Z” axis (i.e. in a third plane), in one example. The geometry of the device may occupy a third plane substantially when an expanded portion along the device is made in order to dilate tissue or to have a particular tortuous curvature specific to a given surgical route through tissue. The degree of curvature along the curvilinear segment is determined by the surgical task and can vary markedly from about 2 degrees to about 175 degrees, or from about 5 degrees to as much as 160 degrees in any plane, in non-limiting examples. In some non-limiting embodiments, the device may be best suitable for surgically altering tissues below the dermis, for example, for deep tissue dissection.

In some non-limiting embodiments, the device may be provided wherein the angle made by a line drawn along the straight section and a line connecting the distal-most end of the straight section and the proximal-most portion of the curvilinear section is greater than 70 degrees when a Y-axis height of the curvilinear segment is equal to or more than 5.3 mm, in one example.

The total length of the device as measured along its longitudinal axis or from distal tip to proximal end and/or proximal end opening may include at least 7 mm in length, in another embodiment the total length of the device may include at least 8 mm in length, and in yet another embodiment, the total length of the device may include at least 9.9 mm in length. In some examples, the maximum length of the device may exceed 1 meter in total length, for example, for use for sub fascial placement and/or cutting. Portions of the device may include features such as a sharp pointed distal end, a dull rounded-blunted distal end, a reverse cutting configuration, a conventional cutting configuration, or various swage attachment configurations to accommodate a flexible wire filament attachment, for example. Moreover, the device may be formed of thermoplastic or metal material in non-limiting embodiments to increase strength by increasing cross-sectional area.

A standard cutting sewing needle includes a sharp edge along its concave side. A reverse cutting needle has a sharp cutting edge along the outer, or convex, side of the needle. Needles can be made to cut, as described herein, by creating a sharp edge along their side(s). A swage includes a proximal part of a stitching needle, wherein the thread attaches into a hollow area within the metal so that the thread appears to be continuous with the needle, for example.

When cutting deep tissue by percutaneous means a torque device or needle with suitable geometry for the designated procedure is used. The user may grasp the device at or near its proximal end by a portion of the curvilinear segment. The distal end of the device may include a sharp or blunted end and may be pushed through skin at a point designated “A”, in one example. The device may then be guided to the target location designated “B”, for example, by advanced imaging, such as by sonography, in a non-limiting embodiment. Between skin points A and B, the tip of the device is passed beneath the desired target tissue whereupon a torque is generated by the user to utilize the proximal end to direct the straight segment toward the skin (at “B”), in one non-limiting embodiment. The entire length of the straight segment may be placed through the tissue. The curvilinear segment reaches the tissue following penetration by the straight segment. The curved segment(s) of the device can be used to further encourage the tip of the device to move along a preferred path by its degree of curvature. The path of the device is, therefore, influenced by multivariate forces not to exclude the densities of the surrounding tissue (e.g. fat, muscle, tendon, ligament, fascia etc.), the force vector produced by the user and the curvature of the device that urges the more proximal portion of the needle toward a path determined by the path of least resistance of the curvature through relatively elastic tissue. The device can, in some embodiments, be loaded with a suture or other device at points along its length as described herein. The attachment accessories may follow the path of the distal tip in some embodiments. A properly placed flexible filament loop can be used to cut, dilate, deform, debride or sew tissue and may be then retrieved (either through A by backing the device out or through B by pulling the device out through the exit point entirely), for example. Other more advanced derivations of this basic “A to B” technique are also contemplated herein.

In one embodiment the device may include an opening, also referred to as an eye at the proximal end as shown in FIG. 3, in some non-limiting embodiments, the device can track from an entry point “A” to an exit point “B” and can then be removed through the skin at exit “B”. The term “track” or “tracking” as used herein may refer to, in some embodiments, the trajectory of the device passing through the tissue.

In one example, a suture or other filament may be associated with the opening and may traverse the skin at entry point A and may be passed beneath a target anatomic structure to exit point “B”. Once the device is passed through exit point “B” as shown in FIG. 8, exit “B” may then become a new entry point. The device may then be redirected, with the filament still associated with the eye, back to point “A” wherein the device (and the attached suture or other filament) is directed in a more superficial plane i.e. above the target anatomic structure. The suture (or other filament) is now looped around the desired anatomic structure and can be used to cut the target anatomic structure. Alternatively, a knot may be tied percutaneously to effectively sew the anatomy, in some non-limiting embodiments. This particular method embodiment is summarized by the steps shown in FIG. 11, using the distal tip from [A to B then B to A].

Alternatively, the device can be directed to sew over a desired distance. In this technique the device with a trailing filament is placed through an entrance point “A” and then directed through a desired tissue plane. The device is then directed to exit though another location “B” and is removed from the body entirely. The distal tip may then be placed through “B” and then directed to a third point “C” where the device can be removed from the body. The filament now lies in a desired percutaneous plane from point “A” to point “C”, and this process can be repeated at a plurality of entry and exit points as needed, in some embodiments. In this type of percutaneous sewing, the goal is usually to arrive back at point “A” so the filament, in some examples a suture, can be tied to complete the surgery. This technique is summarized by the steps of the distal tip as [A to B to C to X (usually back to A], wherein X includes a point outside the body, in one example.

Another example of a use of the device embodiment provided herein includes a device with the opening at the distal end of the device. An opening, including an eye or other means of attachment for a suture may be placed anywhere along the device however; for the purposes of percutaneous procedures, one embodiment of the device may include an eye toward the distal end. In this embodiment, the suture is at the distal end.

Oftentimes, practitioners are faced with having to retrieve a filament for cutting or sewing, for example, from the end of a device such as a needle in instances when it is impractical for the geometry of the needle to pass completely through the desired anatomical space. In these situations, the distal tip and a portion of the body can be exposed. By exposing the filament with the distal end tip, the practitioner can retrieve the filament and remove the device from the space by the same tract it traversed to enter the space effectively solving the problem of the proximal loaded suture being impassable through an anatomic space.

Another use for the embodiment wherein the eye is placed at the distal end of the device includes the percutaneous passing of suture so as to be looped around an anatomic structure in a different sequence than previously explained. In this sequence a suture can be percutaneously passed from point A to point B. The suture can then be retrieved from the end of the device which can be made to protrude from point B. The device is then removed from (backed out of) point A and then reinserted in point A but redirected more superficially, above the target anatomy, to exit (i.e. protrude through) point B. The suture is again removed from the end of the device and now lies looped around the anatomy beneath point A. In this procedure, the practitioner may be required to remove the suture from the distal end of the device, which may be a sharp end. These actions are usually discouraged in most surgical philosophies to avoid practitioner injury, or exposure. However, a blunt tipped version of the device comprising a distal opening distal eye needle may prevent risk of such injury. These techniques are summarized by the steps of the distal tip as [A to B to A′] with the prime (′) designation indicating a skin point that has changed from entry to exit where the needle reverses course along the same path it had just travelled.

In examples of procedures where a filament is looped around anatomy with the intention to cut, the filament can be pulled against the anatomy and with back and forth motions (such as with a Gigli saw technique) to transect the anatomy. Alternatively, a suture can be used to tie and secure the anatomy, or a dilating component can be used for additional dissection. Infinite configurations of the filament can be created to effectively alter the target tissue for medical use.

In certain embodiments of the invention, in order for the device to have the desired length for deep dissection, the length of the substantially straight section may be equal to or greater than 4.6 mm in length, for example. The substantially straight section may include the higher value of either length from a starting point (e.g. “A”) to the target tissue, as measured in a straight line or the length of the overlying target tissue taking into account factors of tissue elasticity. The length of the straight segment may also accommodate the threshold for optimal detection by advanced imaging. For procedural purposes the user should have an embodiment including a chord length (i.e., a length determined by a distance between the distal tip of a needle and the proximal end of the needle) of the device that is longer than the planned step-wise skin entry and exit points. This is to avoid losing control of the needle, wherein, for example, the chord length is shorter than the distance between point A and point B through which the needle enters and exits, respectively, the skin in one example resulting in the practitioner losing sight, contact, and/or control of the device.

The device includes a substantially straight distal segment but may then include any shape to its distal end, in non-limiting embodiments. In one example, the needle may form a V or U shape or an L shape as abrupt changes in needle geometry may be well tolerated by tough resilient tissue. In other embodiments, a cross sectional surface area of the device may vary from the proximal end to the distal end. In one example, the proximal end may include a larger cross-sectional surface area than the distal end. In another embodiment the cross-sectional surface area may be larger at the distal end than at the proximal end. In yet other non-limiting embodiments, embodiments the cross-sectional surface area my otherwise vary along the length of the device for example, both proximal and distal ends may include smaller cross-sectional surface areas as compared the central portion of the device from end to end. Therefore, in some non-limiting embodiments, the cross-sectional surface area of the proximal end may be equal to the cross-sectional surface area at the distal end. Lastly, the cross-sectional surface area may remain generally consistent from the proximal end to the distal end.

The device may include regions containing various surface treatments. The device may include various cross-sectional shapes, wherein a shape of the cross section may include a triangular shape, a square shape, an ellipse shape, a circle, or an oval cross-sectional shape, among others. In some embodiments, the device or at least a portion thereof may be hollow.

The novel device described herein in various embodiments is described in relationship to a trigger digit procedure where a segment of tissue may be transected by looping a filament around the tissue. However, the device embodiments described herein are likewise suitable, in other embodiments, for tunnel surgeries such as the carpal tunnel, or cubital tunnel when the course of a nerve is accessible to surface-to-surface needling. In some embodiments, the device may also serve to percutaneously repair tissue such as for example, the achilles tendon.

There are many methods for repairing the achilles tendon. Typically, the methods include a suture with or without a synthetic or human graft to increase strength of the repair. The novel device and method embodiments described herein provide an ability to percutaneously repair achilles ruptures providing an approach that safely approximates and secures the achilles while minimizing the complication risks common to open procedures, including infection, wound dehiscence, adhesions, and the complications of general anesthesia.

In some embodiments provided herein, a method for achilles repair may include a sequence such as [A to B then B to A] using a device embodiment with an opening placed at or near the proximal end of the device in one example. This procedure may differ from tunnel release surgeries by way of the needle and suture traversing through instead of around the pathologic tissues. Precision in maneuverability of the device may still depend on the ratio of straight segment to curved segment, in at least one embodiment, and/or the progression of curved segment curvature and/or the stiffness of the device relative to the tissue.

The device embodiments described herein for use in achilles tendon repair provides an ability to robustly approximate the torn ends while limiting any impact to the soft tissues and the surrounding blood supply to the tendon. In one particular embodiment, the device may be placed into a healthy portion of the tendon and then channeled by ultrasound guidance through the tendon and to the tear/rupture site. The device may then be guided into healthy tendon tissue on the other side of the tear and surfaced through the superficial tissues and then the skin. The distance between entry and exit points is less than the chord length of the large needle. A filament (i.e. suture) is then placed into either the distal-end or the proximal end of the device whereupon the device is removed with the trailing suture now spanning the same anatomy traversed by the device.

A second pass of the device may place the device superficial to the first pass and using the same entry and exit points and its trailing suture exits the anatomy by the same path as the other free end of the filament. By way of this sequence in particular, the filaments are positioned in a loop within the achilles tendon providing the adjacent free ends in a position to be joined. Joining of the free ends may occur outside the anatomy, in one embodiment. Two knots, which may include half-hitch knots, in some embodiments, may be thrown in the same direction and may be used to slide the knot to the achilles. Further tightening of the knots closes and repairs the rupture gap. As needed, the knot can still be slid down the suture by pulling the suture ends in opposing directions using a novel method described herein, where one of the ends of the suture is held by an instrument (preferably a thin instrument such as a vascular hemostat, for example) which is placed safely within a tissue plane of the surrounding anatomy. The final knot may be tied in several ways. In one example, a simple knot thrown in a reverse direction may effectively locks all of the knots together, in one embodiment. In one embodiment, sutures having a relatively smooth texture with high tensile strength may be preferable. A second or third filament can be similarly looped about the rupture if needed to approximate the rupture or to provide additional strength.

Ultrasound guidance methods may be used, in some embodiments, to facilitate the guidance of the device embodiments and prevent cutting errantly into adjacent anatomy. For achilles surgeries, suture entrapment of the sural nerve in a percutaneous repair can be a devastating injury leading to painful neuroma and/or loss of sensation to the lateral distal leg and/or to the foot. While visualization of the sural nerve is a well-known technique for advanced sonographers the technique is not instructed as a routine precaution for any known percutaneous achilles repair method. Using the methods described herein, the sural nerve can be observed and even retracted when necessary during the operation. The straight leading portion of the dissecting needle allows advancement of the device through the achilles tendon while observing the sural nerve.

The methods described herein for the device avoids larger incisions that may experience wound breakdown known as dehiscence. The methods may also prevent abscess, keloid and/or granuloma formation which notoriously complicate an estimated 10% of achilles open surgeries. The small punctures (in some instances of about 1.5 mm) of the device into the skin do not routinely require sutures of any kind. There is no other known technique for percutaneous suture placement capable of such small puncture-styled wounds. A predictably stable wound along with the well-approximated tendon can advance the time to active motion and rehabilitation of the tendon which further preserves calf muscle mass and function.

The device is also capable of percutaneously shuttling grafts and “internal braces” into, through and/or beside the achilles to greatly strengthen the repair of a ruptured achilles or to augment and reinforce a pre-rupture condition such as tendonosis with greater than 50% debridement, or fulminant calcific “tendonitis” for example. A structural brace is an internally placed device (in this case, most probably heavy suture) that spans the achilles repair in such a way as to add strength to the repair or to protect the repair during the healing process. These more advanced methods can dramatically decrease rehabilitation time and push the final healing result closer to the complete restoration of the strength of the native tendon and its associated musculature. The grafts and braces are placed through an end of the dissecting needle and guided, by ultrasound, in some embodiments, precisely to the necessary sites of attachment relative to the achilles. Percutaneous suturing techniques, as previously described, can then secure the device or graft.

The device embodiments described herein utilizes the latest ultrasound real time imaging to surgically repair the achilles tendon while decreasing surgical complications and decreasing surgery related costs by reducing the need for prolonged convalescence or formal rehabilitation therapies. Accordingly, the method embodiments described herein for percutaneous achilles tendon repair may be performed as an outpatient such as in a clinic setting, unless a patient's medical condition warrants more advanced positioning or anesthesia care. The cost savings of the office procedure in comparison to hospital costs can be significant with the office procedure being possibly one-twentieth the cost of the hospital-based surgery by contemporary billing, coding and collections data. The cost savings of the percutaneous procedure compared to open procedures can be even more substantial since the wound complication rate is lower and the time to rehabilitation (and/or back to the work place) is significantly shorter with some patients not requiring the expense of formal physical therapy at all.

One embodiment of the device used for achilles repair may be 13 cm in length with a straight segment of approximately 5.5 cm. The curved portion elevates along the Y-axis (from the X-axis straight segment) to approximately 5 cm forming a chord length from the proximal-most tip of the needle to its distal most tip, of approximately 10 cm. An eye is positioned at the proximal most portion of the curved segment for the coupling of a stout suture suitable for achilles repair. The device can be made inexpensively using stainless steel, in some non-limiting embodiments, wherein properties of the steel such as stiffness/elasticity, and toughness can be adjusted according to the surgeon's preference. The device may be handled by a small handheld vice grip due to the size of the device and the density and toughness of the tissue that it negotiates.

In one embodiment, a method for percutaneous repair of a tendon rupture may be provided including inserting the distal end of the device by traversing the skin of a patient at a first target area (A), of the patient that overlies normal tendinous tissue on one side of the rupture, wherein a first end filament attached to the proximal end of the device traverses the skin at the first target area (A), passing the device into the tendon at point (A) and directing the device toward the deep half of the tendon and simultaneously toward the rupture area by manipulating a proximal end of the device, passing the device through the rupture area and then into the deep half of the tendon on the other side of the rupture, penetrating normal tendon on the other side of the rupture and then directing the distal end of the device superficially to the skin at point (B);

removing the device from the tissue at point (B) whilst the filament remains within the anatomy along the course that the device traversed, removing the filament from the device; reinserting the distal end of the device at point (A) and directing the distal end to remain within the superficial half of the tendon; passing the device through the rupture area and then into the superficial half of the tendon on the other side of the rupture; penetrating normal tendon on the other side of the rupture and then directing the distal end of the device superficially to the skin at point (B); removing the device at point (B) and further removing the device from the filament which now lies looped through the tendon with both free ends of the filament exiting through point (B); tying the free ends of the filament together by two half-hitch knots thrown in succession and in the same direction; pulling on one end of the filament such that the two half-hitch knots slide beneath the skin and toward the tendon whilst the rupture gap is repaired by the tendinous ends being compressed together; making a third half hitch knot with the free ends of the filament, the third half hitch being thrown in the opposite orientation of the first two knots; manually sliding the third half hitch knot to lie against the first two and then pulling the free ends of the filament in opposing directions to tighten the knots against the tendon; and cutting the free ends of the filament so that the entirety of the filament lies below the skin.

In another embodiment, a method for directing the device between the A1 pulley and the flexor tendon sheath using a waypost needle may be provided including: a waypost needle is viewed in long axis by an ultrasound machine and then placed into a hand in a perpendicular orientation to a flexor tendon, and then through the proximal-most portion of the A1 pulley ligament, a dissecting needle is then placed through a patient's skin in long axis and in line with the longitudinal course of the affected flexor tendon toward the waypost needle which is viewed in short axis at the same. The dissecting needle is advanced to abut the underside (i.e. deep side) of the waypost needle wherein the dissecting needle is forced into the plane between the A1 pulley and the flexor tendon. The trilaminar structure of the A1 pulley is seen by the ultrasound machine to lie above (superficial to) the dissecting needle and to separate from the underlying flexor tendon as the dissecting needle expands the potential space between the A1 pulley and the flexor tendon, the dissecting needle is then advance by ultrasound guidance to the distal most end of the A1 pulley, and the waypost needle is removed.

Turning to the drawings, FIGS. 1-2B include side views of various non-limiting device embodiments as described herein. FIG. 1 includes a device 100 having a proximal end 102 and a distal end 104, a sharp distal tip 106 disposed at the distal end 104. The device 100 also includes a curvilinear section 108 between the distal end 104 and the proximal end 102, and a substantially straight segment 110 extending between the sharp distal tip 106 and the curvilinear section 108. Near the most proximal end of the device 100 an opening 112 is provided for receiving a trailing filament, in one non-limiting embodiment. In another non-limiting embodiment, in addition to the opening 112, an opening near the distal end 104 can also be made to provide two options for filament coupling.

FIG. 2A includes another embodiment of a device 200, including a proximal end 202, a distal end 204, including a blunted distal tip 206 at the distal end. A curvilinear section 208 is provided between the proximal end 202 and the distal end 204, and a substantially straight section 210 is disposed between a blunted distal tip 206 and the curvilinear section 208, for example. An opening is provided at the proximal end 212 in one non-limiting embodiment.

FIG. 2B provides an embodiment of a device 300 including a proximal end 302, a distal end 304, and a blunted distal tip 306 at the distal end 304. Near the distal end 304, an opening 312 for receiving a filament, in one example, is provided. The opening 312 may be provided along the length of the device 300 at any point. Between the proximal 302 and the distal end 304, is provided a curvilinear section 308. Between the blunted distal tip 306 and the curvilinear section 308, is provided a substantially straight distal section 310, in one embodiment.

As shown in the device embodiment 400 of FIG. 3, an arc and length of the curvilinear section 408 may vary according to the preferred route through the anatomy, the tissue type, the patient, or other factors. FIG. 3 also shows an opening 412 extending from a proximal end thereof.

Nonlimiting examples of details of the device may include, in one embodiment, the substantially straight segment may include a length of between 1-2 cm, in one example the length may be 1.7 cm, and the curvilinear section may include a length of between 2 cm-3 cm, in one example, the length may be 2.5 cm. In one non-limiting embodiment, the length of the device 100, 200, 300, 400, from proximal to distal end may be approximately 4.2-4.5 cm. In one particularly non-limiting example, the total length may be 4.2 cm, and the chord length of the device may be 3.5 cm.

FIG. 4 shows a human hand 10. The hand 10 depicts a ring finger 12 in a pathologic position of flexion as is often clinically observed in the condition of surgical trigger digit. The hand 10 further includes a flexor tendon 85, an A1 pulley 95, and a fourth metacarpal bone 86 that lies beneath the flexor tendon 85 and provides attachments for the A1 pulley 95 which encircles the flexor tendon 85.

FIG. 5 is a side view of FIG. 4 demonstrating the A1 pulley 95, flexor tendon 85, metacarpal bone 86 and the device 100. The A1 pulley 95 lies on the ventral side of the 4th metacarpo-phalangeal joint 12. The flexor tendon 85 lies on top of the bone 86. The device 100 has a tip or distal end 106 that has penetrated the skin 11 and subcutaneous tissues 13 to pierce an opening at the entrance of the proximal A1 pulley 95. Alternatively, device 200, 300 (not shown in FIG. 5) having a blunt tapered distal end 206, 306 would require a prepared puncture at point A before being pushed to the proximal end of the A1 pulley 95. A sharp instrument such as a hypodermic needle could be used to prepare a puncture at point A, and also puncture between the flexor tendon 85 and the A1 pulley 95, in a non-limiting example prior to the use of the device 200, 200 embodiments.

FIG. 6 shows the straight segment of the device 100 positioned between the flexor tendon 85 and the A1 pulley 95. The distal tip 106 is positioned at the distal end of the A1 pulley 95. The opening 112 for receiving a filament of the device 100 and a part of the proximal end 102 is still positioned outside the skin 11. The skin 11 and subcutaneous tissues 13 have enough elasticity to accommodate movement of the device 100 as shown in FIG. 5 and FIG. 6. Alternatively, device 200, 300 having a blunt end 206, 306 could be used to bluntly dissect or traverse in between the A1 pulley 95 and the flexor tendon 85 with the advantage of having a decreased chance of penetrating into either tissue during the transit between them. In this way, the blunt tip embodiment 200, 300 has certain benefits.

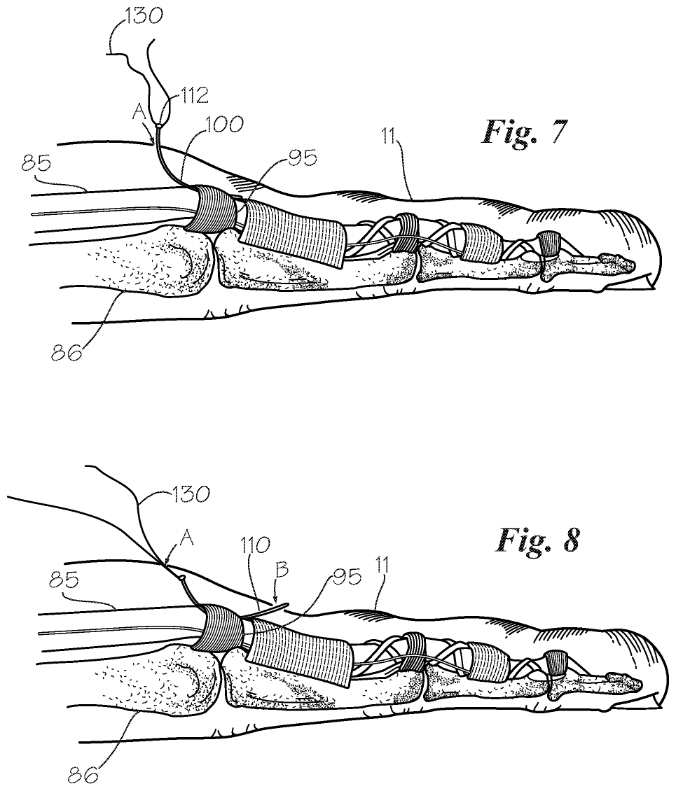

FIG. 7 shows a filament 130 being placed through the opening 112 of the device 100 while the device 100 remains in the same position as shown in FIG. 6. The filament 130 can be placed through the opening 112 at any time before the opening 112 traverses the skin 11.

FIG. 8 shows the distal end of the device 100 being directed upwardly toward exit point “B” and piercing the skin 11 and exiting at point “B”. The device 100 still lies between the flexor tendon 85 and the A1 pulley 95 and a small portion of the substantially straight portion 110 has now exited through point B. Alternatively, for device embodiment 200, 300, a sharp instrument, such as a hypodermic needle, may be used to pierce the skin at point B to allow the device 200, 300 to exit through point B.

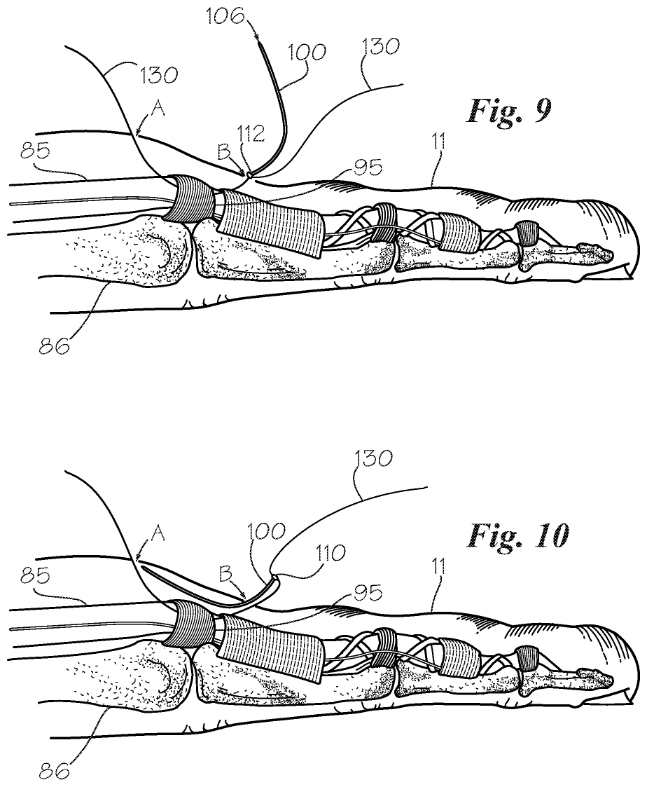

The user is able to grasp the device 100 shown in FIG. 9 at or near its distal tip 106 to pull it completely through the skin 11. The chord length of the device 100 is greater than the length between entry “A” and exit “B”, in one example. For this embodiment, the length between entry A and exit B as shown may be less than 3.5 cm.

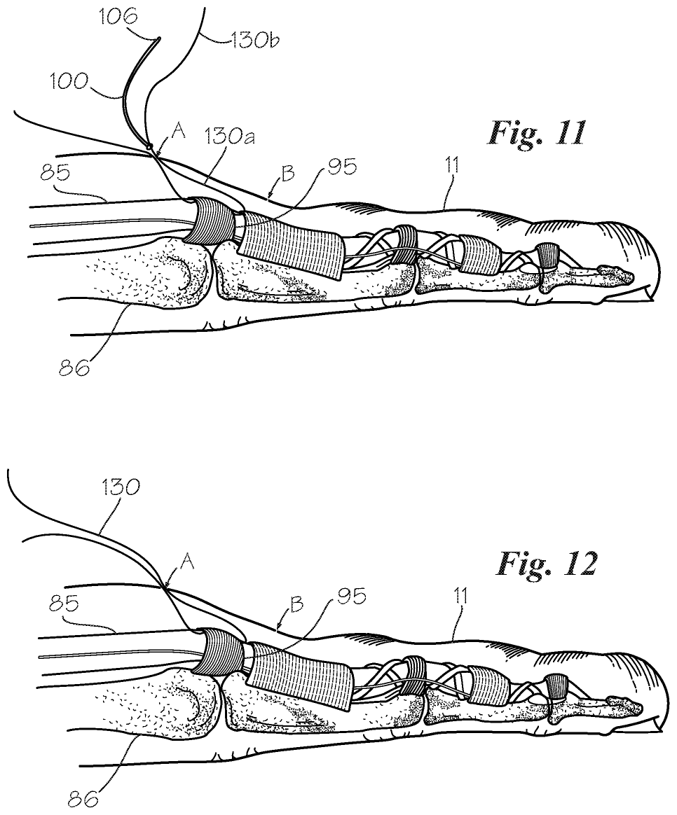

FIG. 9 demonstrates the device 100 being completely pulled through exit point “B” and trailing behind the device is a filament 130, which traverses the first opening A and second opening B and extends through the opening 112 of the device 100. The filament 130 now lies outside the skin 11 at both points, entry A and exit B, and is positioned between the A1 pulley 95 and the flexor tendon 85. The device 100 still retains the suture 130. As shown in FIG. 10, the distal tip 106 is directed back through point B as new entry point. Alternatively, the device 100, 200, 300 could be removed from the filament 130 and then pushed through point A with the filament overlying point A, filament 130 being placed through the opening 112, 212, 412 to begin placement of a loop of filament at the proximal edge of the A1 pulley 95.

FIG. 10 shows the substantially straight portion 110 of the device 100 being directed between the skin 11 and the A1 pulley 95 and being directed toward entry point “A” to begin placement of a loop of filament at the distal edge of the A1 pulley 95.

Notably in this step as in the previous steps, a blunt end device embodiment 200, 300 could be used as desired. Alternatively, the device 100, 200, 300 could be placed again through point A.

FIG. 11 shows the device having been moved superficial to the skin through point “A”, the filament 130a and 130b now distally looped around the A1 pulley and with both filament ends exiting through point “A”. The figure shows the device 100 (and distal tip 106) with filament 130b still retained at the opening 112 of the device.

FIG. 12 shows the device 100 has been pulled through the skin 11 completely through point “A” with the filament 130 now making a loop around the A1 pulley 95. In an alternative embodiment, any device embodiment, 100, 200, 300, 400 could be inserted through point A (see FIG. 10), transit between the skin 11 and the flexor tendon 85 so as to exit through point B [ A to B then with 130a A to B].

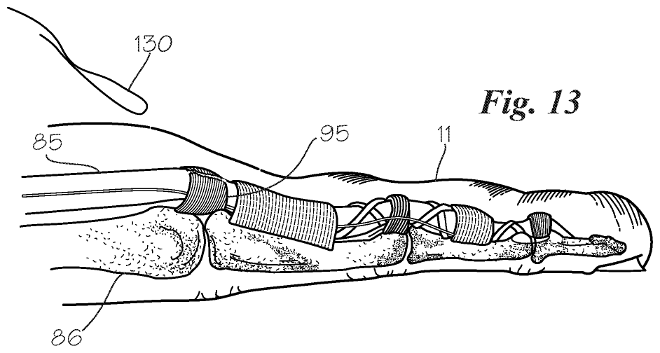

FIG. 13 shows the filament 130 having transected (i.e., cut through) the A1 pulley 95 and the filament 130 is removed completely from the anatomy through point A. In an alternative embodiment, the filament 130 could be looped in such a way that the filament 130 exits through point B. In other non-limiting embodiments, the filament may be used to partially cut the tissue, or in other embodiments, to simply move or displace the tissue (whether cut or uncut).

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements. Moreover, all ranges disclosed herein are to be understood to encompass any and all sub-ranges subsumed therein. As a non-limiting example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 7.

It should be borne in mind that all patents, patent applications, patent publications, technical publications, scientific publications, and other references referenced herein are hereby incorporated by reference in this application in order to more fully describe the state of the art to which the present invention pertains.

It is important to an understanding of the present invention to note that all technical and scientific terms used herein, unless defined herein, are intended to have the same meaning as commonly understood by one of ordinary skill in the art. The techniques employed herein are also those that are known to one of ordinary skill in the art, unless stated otherwise. For purposes of more clearly facilitating an understanding the invention as disclosed and claimed herein, the following definitions are provided.

While a number of embodiments of the present invention have been shown and described herein in the present context, such embodiments are provided by way of example only, and not of limitation. Numerous variations, changes and substitutions will occur to those of skill in the art without materially departing from the invention herein. For example, the present invention need not be limited to best mode disclosed herein, since other applications can equally benefit from the teachings of the present invention. Also, in the claims, means-plus-function and step-plus-function clauses are intended to cover the structures and acts, respectively, described herein as performing the recited function and not only structural equivalents or act equivalents, but also equivalent structures or equivalent acts, respectively. Accordingly, all such modifications are intended to be included within the scope of this invention as defined in the following claims, in accordance with relevant law as to their interpretation.

What is claimed is:

1. A non-invasive surgical trigger finger repair device comprising:
a proximal end comprising a component for coupling to a filament;
a blunted distal end;
an elongated straight section extending from the distal end toward the proximal end; and
a curvilinear section between the elongated straight section and the proximal end;
wherein manipulation of the device and targeting of the distal end occurs by movement of the curvilinear section and/or the proximal end.

2. The non-invasive surgical trigger finger repair device of claim 1, wherein the length of the device is greater than at least 9.0 mm.

3. The non-invasive surgical trigger finger repair device of claim 1, wherein the length of the device is greater than at least 9.9 mm.

4. The non-invasive surgical trigger finger repair device of claim 1, wherein the curvilinear section comprises a length of at least 5.0 mm.

5. The non-invasive surgical trigger finger repair device of claim 1, wherein the curvilinear section comprises a length of at least 3.0 mm.

6. The non-invasive surgical trigger finger repair device of claim 1, wherein the elongated straight section comprises a length of at least 4.0 mm.

7. The non-invasive surgical trigger finger repair device of claim 1, wherein the elongated straight section comprises a length of at least 4.6 mm.

8. The non-invasive surgical trigger finger repair device of claim 1, wherein the curvilinear section elevates along the Y-axis at a minimum of 5.0 mm.

9. The non-invasive surgical trigger finger repair device of claim 1, wherein the curvilinear section elevates along the Y-axis at a minimum of 5.3 mm.

10. The non-invasive surgical trigger finger repair device of claim 1, wherein the curvilinear section further comprises at least a 70 degree curve.

11. The non-invasive surgical trigger finger repair device of claim 1, wherein the curvilinear section further comprises at least a 50 degree curve.

12. The non-invasive surgical trigger finger repair device of claim 1, wherein the distal end further comprises an aperture for receiving a filament.

13. The non-invasive surgical trigger finger repair device of claim 1, wherein the straight section is disposed in a first plane, and wherein the curvilinear section is in a second plane.

14. The non-invasive surgical trigger finger repair device of claim 8, wherein the first plane and the second plane are different.

15. The device of claim 10, wherein the proximal end comprises an aperture for receiving a filament.

16. A method for percutaneous tissue repair, comprising:
inserting a distal end of the device of claim 17 by traversing a skin of a patient at a first target area (A) of the patient, wherein a first end of a filament attached to the proximal end of the device traverses the skin at the first target area (A);
passing the device through a tissue of the patient by manipulating a proximal end of the device;
removing the distal end of the device from the patient by manipulation of the proximal end of the device causing a torque to the device, such that the distal end of the device exits the skin of the patient at a second target area (B) and a first end of a filament exits the skin of the patient at the second target area (B);
re-inserting the distal end of the device into the second target area (B);
passing the device through a tissue of the patient such that the filament loops around a target tissue; and
removing the distal end of the device from the patient by manipulation of the proximal end of the device causing a torque to the device, such that the distal end of the device traverses exits the skin of the patient at target area A and a first end of a filament traverses exits the skin of the patient at target area A.

17. The method of claim 16, further comprising manipulating a first and second ends of the filament, so as to sever the target tissue.

18. The method of claim 16, further comprising manipulating a first and second end of the filament, so as to move the target tissue from a first position to a second position.

19. A method for treating trigger finger by A1 pulley, comprising:
inserting a distal end of a device having a distal end and a proximal end into a hand of a patient at a first target area (A) of the patient, wherein the proximal end comprises an opening for receiving a filament, the device further comprising an elongated straight section extending from the distal end toward the proximal end, and a curvilinear section between the elongated straight section and the proximal end;
manipulating the device, such that the distal end passes through a skin of the user at a first point (A), between a proximal margin of an A1 pulley and a flexor tendon of the hand by movement of the curvilinear section and/or manipulation of the proximal end;
traversing a length of the A1 pulley with the straight section of the device,
applying a torque to the curvilinear end of the device to direct the distal end of the device toward a point (B) on the skin distal to the A1 pulley;
driving the distal end through the skin at the point (B);
removing the device and the trailing filament through the skin at point (B), such that the filament traverses point (A), the space between the flexor tendon and the A1 pulley, and point (B);
reinserting the distal end of the device through point (B) and directing the device between a subcutaneous tissue and the A1 pulley ligament;
directing the distal end of the device through the skin at point (A) by manipulating the curvilinear end, generating a torque, such that the filament loops around the A1 pulley;
removing the distal end of the device from the patient such that the trailing filament forms a loop around the A1 pulley;
transecting the A1 pulley ligament by pulling the trailing filament through the A1 pulley ligament; and
removing the device through point (A).

20. The method for A1 pulley release for the treatment of trigger finger of claim 21, wherein when the trailing filament forms a loop around the A1 pulley, and the distal end of the device is removed from the patient, the trailing filament enters and exits from point (A).
